(19) United States
(12) Patent Application Publication
(10) Pub. No.: US 2010/0040196 A1
(43) Pub. Date: Feb. 18, 2010

(54) X-RAY GENERATOR FOR ACHIEVING STEREOSCOPIC IMAGING EFFECT AND MEDICAL X-RAY DEVICE USING THE X-RAY GENERATOR

(76) Inventors: Yingguang Zhang, Guangdong (CN); Yanfang Li, Guangdong (CN); Taili Zhou, Guangdong (CN)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/531,072

(22) PCT Filed: Apr. 2, 2008
(86) PCT No.: PCT/CN2008/070664

§ 371 Date: Sep. 14, 2009

(30) Foreign Application Priority Data
Mar. 14, 2007 (CN) 200710027152.0

(51) Int. Cl. A61B 6/02

(52) U.S. Cl. 378/42; 378/41

(57) ABSTRACT

The present invention provides an X-ray generator for achieving stereoscopic imaging effect and an X-ray device using the X-ray generator (101) as the X-ray source. The X-ray generator (101) can emit X-rays alternately from two positions the space of which conforms to the requirement for achieving stereoscopic imaging effect of the human beings.

### TECHNOLOGY FIELD

The present invention relates to an X-ray generator and an X-ray device, especially to a medical X-ray generator and a medical X-ray device.

### BACKGROUND ART

### I. X-Ray Generators in the Art

The X-ray tube has come into our life for almost one hundred years, and the X-ray has been applied to various fields of social life, such as industrial radiography, chemical analysis, jewelry appraise, security inspection in airport and railway station, X-ray examine in medical treatment, CT scan device invented in 1970s, Digital Subtraction Angiography, digital X-ray perspective apparatus and Direct Radiography. However, the working principle of the X-ray tube doesn't change, which is that electron beam bombards heavy metal to generate X-ray.

The earliest X-ray tube is cold cathode, its working principle is that: an electrode called anode is joined in the discharge path of the discharge tube, and voltage is applied on this anode, the X-ray is generated when the discharge tube discharges. The electrode is made of heave metal, such as molybdenum, tungsten, nickel and cobalt,

The existing and widely used X-ray tubes have two categories, which are fixing anodes tube and rotating anodes tube.

The tubes which are used in miniature X-ray machine and industrial X-ray machine are fixing anodes tube, the majority of the packaging mode is glass tube shell, and a minority is metal tube shell. The advantages as following: the anode is fixed, the structure is simple, and the manufacturing cost is low. And the disadvantages is that the power is low and the quantity of X-ray is few so it can only be used in miniature X-ray machine, CT machine or DSA machine can't apply this kind of tube. The fixing anode tube comprise filament, anode target which is made of heavy metal and glass tube shell or metal tube shell which is pumped vacuum.

Another kind of X-ray tube is a rotating anodes tube. The rotating anodes tube is high performance tube, which is invented after high power X-ray machine. It can use higher current to work under small focus. Viewed from the metal heat dissipation, metals all have thermal resistance when they transfer heat, so the anode target may produce high temperature when the tubes work under small focus and high current. When the electron beam bombards one place continuously, the target may melt soon. If we make the target move, the electron beam may change bombardment place, so the tube shell can work under higher current, the heat which generated in target can transfer into the inner of the metal to prevent melting.

The main difference between the structure of the rotating anodes tube and fixing anodes tube is anode, the anode of rotating anodes tube is a disk with a shaft, and anode rotates by rotating magnetic field which is generated by motor stator coil which is mounted outside the tube core. The anode shaft actually is a rotor of hysteresis motor, nothing but it is sealed in vacuum. The function of the rotating anode is that it can generate X-ray by bombarding of electron beam.

The cathode of the rotating is a direct-heating vacuum tube cathode, its function is that it can generate electron beam to bombard anode. Its glower is made of tungsten, there is a beam current cover which can control the angle and directional of the electronic ray and it is used to control the size of the focus.

There is a kind of bifocal tube, the kind of bifocal tube has two groups glower cathode, one which can generate big focus has high power and can generate strong X-ray, another one which generate small focus has low power can be used to form more clear image. But there is no difference in position, the big focus covers small focus, a pair of data which accord with the requirements of human stereo vision can't be gained to form stereo vision effect image.

In addition, X-ray tube shell contains circulating cooling system and other assistant system. But however, a pair of data which accord with the requirements of human stereo vision can't be gained to form stereo vision effect image

### II. Relief Television Technology in the Art

In the European renaissance period, the research and practice of painting perspective and sculpture art indicated, only provide relatively independent images to the eyes, they can gain genuine stereo vision when the binocular disparity is recovered. In the early stage, binocular stereo vision technology applied the method that observe stereo image by colored spectacles and stereoscope. At the 16^{th} century, people began to apply different colors to protract images which have regular difference for left and right eyes, and then observe the images by filter to generate stereo vision. The stereoscope which has arisen at the end of 17^{th} century to the early of 18^{th} century, provides independent visual channel, generates strong stereo vision. This kind of stereoscope is still efficiency means to observe stereo vision. In the 19^{th}, scientists tried to observe stereo image without using assistant device, but they failed.

At the end of the 19^{th} century, people try to use the film technique to exhibit motive stereo vision. First adopt two cameras to simulate human binocular to screen, then the film is projected to the screen by bioscope through polarizing filter, audience can observe the motive stereo vision by polarizing filter. The film technique is still used today.

The early of 20^{th} century, after appearing of the television technique, people begin to develop relief television, the traditional stereo display methods which are used in observing still image or film image nine tenths are used in relief television technique.

In the early period of monochrome television, more successful relief television is that two television cameras shoot the images and the images were transferred to two televisions by two independent video channels. A deflection board was mounted on each screen of the television, so this relief television system can gain better stereo images through deflection glasses. This kind of double-channel deflection image separating relief television technique is still a better relief television system.

In 1950s, color television develop to the practical stage, the anaglyph stereoscopic television technique began to be applied. to relief television. The basic method is that two cameras which are mounted filters at the front of the lens are used to screen the same scene image; the audiences can see two images which are different colors from the color television screen, so the audiences can see relief television image through corresponding filter. This kind of relief television imaging technique was the rage in the relief television technique field because of its good compatibility. But it had obvious problems: the color information was made great loss; the cross color may cause disturb; and it may cause visual fatigue because of the inconsistent incident spectral of the left and right eyes.

With the appearance of the ceramic optical switch at the end of 1970s, people can make optical switch glasses, so the time-division relief television technique appears. The time-division relief television technique is to adopt the odd field and even field of color television signal to code relief television. In the early of 80s, Toshiba Corporation developed time-division relief television projection machine. In 1985, Panasonic Corporation developed time-division liquid crystal glasses relief television. At present, the helmet watching equipment which has double screens display has very perfect stereo vision effect. Tsinghua University has developed the new-type liquid crystal light valve glasses and time-division liquid crystal glasses relief television in 2001.

At present, the time-division relief television technique is mature comparatively. It has obvious advantage: provide lively color stereo image; the image is stabilization and non-flickering when the television field frequency is much higher; compatible to actual color television system and computer display system; the transition to digital television system is easy.

In 2000, the first real time stereo display system appears in China. Multiple images are played by VCD, so the fierceness stereo image can be gained by wireless infrared glasses. The existing signal source two-dimensional image can be transferred into three-dimensional image on display by stereo display system. But this kind of stereo image effect still rests on using the method of optics or signal processing to transfer the image.

The new-type stereo vidicon and stereo display unit are being developed. The new-type stereo vidicon has double lens, the technology of comprehensive computer, measurement and control, image treatment, and its shooting process conforms to the requirement for achieving stereoscopic imaging effect of the human beings. The new-type stereo display unit imports left and right images, adopts optics technique and can project the left and right images to eyes in accurate parallax. So people can watch stereo image on screen directly without glasses. In the 3D alliance establishment meeting in Tokyo in 2003, Sanyo showed the display on which stereo image can be seen without glasses; Sony showed the shooting and playing of stereo image system. But the stereo effect of the products above is limited by the observing angle and distance.

The basic principle of relief television technique

In the view of the human vision experience, two eyes observe the vision signals, the stereo feeling can be gained; the people of single eye can also gain stereo feeling by observing scene and object. The two cases correspond to the binocular stereo vision and monocular stereo vision. The former one is the basic of relief television; the latter one is gained by experience.

### Binocular Stereo Vision

Relief television generates stereo image by stereo vision characteristic of human eyes. When human watch the world around, they can not only the width and height of the objects, but also the depth, the distance between objects and objects or looker. The reason for generating the three-dimension vision characteristic is that: people observe objects by two eyes, and the space of the visual axis of two eyes is about 65 mm; the left and right eyes receive different images when they watch the object of a certain distant, so the brain generates stereo vision by colligating the information of the two images through the movement and adjustment of eyeball. When people observe objects by right or left eye, the image shift feeling is called parallax. As shown in FIG. 15.

In FIG. 15, if there are two identical vidicons, the planes of two images are in the same plane Q, the coordinate axis of two vidicons are parallel, and the X axis are superposition, the space of two vidicons is baseline B in the direction of X. The two projection points G_{L} and G_{R} which is projected by feature point in scene on the two image planes are called conjugate pairs, that is to say one is the correspondence of another one. After the two images overlapping, the position shift X_{L}-X_{R} of the conjugate pairs is parallax. Supposing the origin of the coordinate system is on the center of left lens, by similar triangle:

X/Z=X_{L}/F and (X−B)/Z=X_{R}/F
obtained: Z=BF/(X_{L}-X_{R}) 1.

So, the depth of the objects is restored by parallax, the bigger the parallax is, the nearer the distance between object and lens. The stereo image pair is gained by stereo vidicon of double lens.

### The Realization Mode of Relief Television

The realization modes of relief television mainly have two kinds: One kind is that two images of a pair of parallax signals appear on the screen at the same time, and then the stereo vision is gained by seeing the two images, such as dual channel polarizing image separating relief television technique and complementary color stereo image separating television technique. Another kind is that two images of a pair of parallax signals appear on the screen alternately, and then the stereo vision is gained by seeing the images at different time, such as time-division relief television technique.

### III. The Medical X-Ray Equipment in Existing Technique

The medical X-ray diagnosing and treating equipments are made of X-ray tube of existing technique, such X-ray digital subtraction angiography machine, digital stomach and intestines machine, digital X-ray perspective machine, and digital direct radiography system. They all use single X-ray source to form plane image without stereo feeling.

Moreover, there are double sources CT machine and double C arms DSA machine. The purpose of using double tubes for double sources CT machine is to improve the scanning speed not to forming stereo vision; the purpose of using double tubes for double C arms DSA machine is to add a projective angle, because of without fixed ubiety for double C arms, the two images can't form a pair of data which conform to the requirement for achieving stereoscopic imaging effect of the human beings to generate stereo effect image.

With the development of modem medicine, the understanding of the spatial position and relation of human tissues, structure is necessary. Therefore the three-dimension reconstruction techniques basing on computer technology are developed, such as CT three-dimension reconstruction technique, MRA three-dimension reconstruction technique, DSA three-dimension reconstruction technique. But these three-dimension reconstruction techniques can't provide real time dynamic stereo images, so doctors only rely on single point X-ray source which is provided by the existing X-ray tube and operate through plane image. In the situation, doctors can only differentiate up and down or left and right, can't differentiate forward and back. It brings risk for interventional procedure, especially neural interventional, cardiac interventional and peripheral interventional.

As a conclusion, the X-ray tubes in exiting technology are very mature and exactitude, but they can only provide single source and form plane image; they can't provide a pair of data which conform to the requirement for achieving stereoscopic imaging effect of the human beings and form stereo vision effect, Although the medical X-ray equipments are also mature and exactitude, they can't form real time dynamic stereo vision effect images.

Obviously, the existing X-ray tubes can't achieve this request, developing a new X-ray generator which can provide real time dynamic stereo vision effect. This kind of medical X-ray equipment can generate stereo vision. In the perspective mode, interventional physician can see tridimensional skull as crystal, blood vessel and bones. The risk of operation is low.

### CONTENTS OF THE INVENTION

The present invention provides an X-ray generator for achieving stereoscopic imaging effect and a medical X-ray device for achieving stereoscopic imaging effect using the X-ray generator as key parts.

The purpose of the present invention is realized as follows:

An X-ray generator for achieving stereoscopic imaging effect and an X-ray device with stereoscopic imaging effect are provided, the X-ray generator deployed to emit X-ray alternately from two positions the space of which conforms to the requirement for achieving stereoscopic imaging effect of the human beings, and the X-ray device use the X-ray generator as the X-ray source.

According to the further feature of the invention, the space of the two positions is close to papillary distance and the space is 40 mm to 90 mm, and the optimum space is 58 mm to 72 mm. The space of the two positions may be fixed or could be adjusted.

According to the further feature of the invention, the X-ray generator comprises at least two X-ray tubes (1) which emit X-ray alternately, and the space (D) of the X-ray tubes (1) is 40 mm to 90 mm, and the optimum space is 58 mm to 72 mm.

According to the further feature of the invention, the X-ray generator for achieving stereoscopic imaging effect comprises two X-ray tubes, thus could be called two-tube type stereo vision X-ray generator, The X-ray generator for achieving stereoscopic imaging effect comprises three X-ray tubes, thus could be called three-tube type stereo vision X-ray generator.

However, no matter how many X-ray tubes are applied, only when the space of the two tubes which emit X-ray alternately accord with the requirements of human stereo vision, a pair of data which accord with the requirements of human stereo vision can be gained, and is independent and associated, and relief television technique or vector-graph technique is applied to form stereoscopic imaging effect.

The X-ray generator for achieving stereoscopic imaging effect comprises cathode, anode, deflection electrode, and at least two anode focuses; the high energy electron beam which is emitted from cathode and controlled by deflection electrode hits the different anode focuses to emit X-ray alternately. The space of different anode focuses is 40 mm to 90 mm; the optimum space is 58 mm to 72 mm. The type of X-ray generators for achieving stereoscopic imaging effect in the present invention can be designed into many different detailed structures, for example, in accordance with the number of anode focus, they can be divided into single-focus type, double-focus type, three-focus type and so on; in accordance with the number of anode, they can be divided into single-anode type, double-anode type, three-anode type and so on; in accordance with anode moving or not, they can be divided into fixing anodes and rotating anodes.

However, no matter how many X-ray tubes are applied, only when the space of the two tubes which emit X-ray alternately accord with the requirements of human stereo vision, a pair of data which accord with the requirements of human stereo vision can be gained, and is independent and associated, and relief television technique or vectorgraph technique is applied to form stereoscopic imaging effect.

Further, the X-ray generators for achieving stereoscopic imaging effect have three anode focuses, which are deployed to emit X-ray alternately from two or three anode focuses. When the space of any two focuses of one-tube and three-focus X-ray generators for achieving stereoscopic imaging effect accords with the requirements of human stereo vision, data which accords with the requirements of human stereo vision can be gained and forms stereoscopic imaging effect. In this way, one-tube and three-focus X-ray generators for achieving stereoscopic imaging effect can provide three different angles stereo vision image without moving the X-ray generators.

The X-ray generators for achieving stereoscopic imaging effect have at least two cathodes, the high energy electron beam from each cathode hits the corresponding anode focuses alternately, and the X-ray is emitted from anode focus alternately. In accordance with the number of cathode, they can be divided into single-cathode type, double-cathode type, three-cathode type and so on.

The present invention also provides X-ray device which applies X-ray generators for achieving stereoscopic imaging effect, especially medical X-ray device for achieving stereoscopic imaging effect.

The medical X-ray device for achieving stereoscopic imaging effect applies X-ray generator for achieving stereoscopic imaging effect as the X-ray source.

Further, the medical X-ray device for achieving stereoscopic imaging effect includes a digital subtraction angiography for achieving stereoscopic imaging effect, which applies X-ray generator for achieving stereoscopic imaging effect as the X-ray source for digital subtraction angiography.

The medical X-ray device for achieving stereoscopic imaging effect includes a digital gastrointestinal apparatus for achieving stereoscopic imaging effect, which applies X-ray generator for achieving stereoscopic imaging effect as the X-ray source for digital gastrointestinal apparatus.

The medical X-ray device for achieving stereoscopic imaging effect includes a digital X-ray perspective apparatus for achieving stereoscopic imaging effect, which applies X-ray generator for achieving stereoscopic imaging effect as the X-ray source for digital X-ray perspective apparatus.

The medical X-ray device for achieving stereoscopic imaging effect includes a direct radiography (DR) for achieving stereoscopic imaging effect, which applies X-ray generator for achieving stereoscopic imaging effect as the X-ray source for digital direct radiography.

The X-ray generator for achieving stereoscopic imaging effect is not only used as X-ray source of medical X-ray generator for achieving stereoscopic imaging effect, can also be used in industrial radiography, jewelry appraise, security inspection in airport and railway station, X-ray examine in medical treatment

The present invention provides an X-ray generator for achieving stereoscopic imaging effect and an X-ray device using the X-ray generator (101) as the X-ray source. The X-ray generator (101) can emit X-rays alternately from two positions the space of which conforms to the requirement for achieving stereoscopic imaging effect of the human beings, so a pair of X-ray image data which conforms to the requirement for achieving stereoscopic imaging effect of the human beings can be gained. The pair of data is treated by computer data processing system to form stereo image in stereo image display system by relief television technique and stereo film technique. By the medical X-ray generator for achieving stereoscopic imaging effect in present invention doctors can observe real time dynamic stereo vision effect image, it is very convenience for doctors in diagnosing and operating.

### BRIEF DESCRIPTION OF THE FIGURES

FIG. 1 is a perspective view illustrating structure of double-tube X-ray generator for achieving stereoscopic imaging effect;

FIG. 2 is a perspective view illustrating structure of double-tube and double-window type X-ray generator for achieving stereoscopic imaging effect. The difference from FIG. 1 is that every X-ray tube emits X-ray from corresponding window;

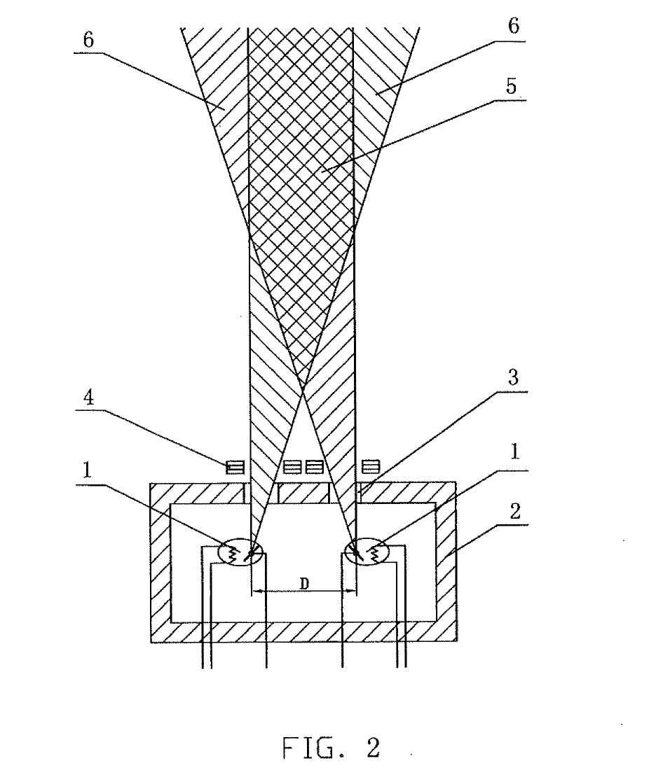

FIG. 3 is a perspective view illustrating structure of independent double-tube type X-ray generator for achieving stereoscopic imaging effect. The difference from FIG. 2 is that every X-ray tube which the shielding case shields emits X-ray from corresponding window;

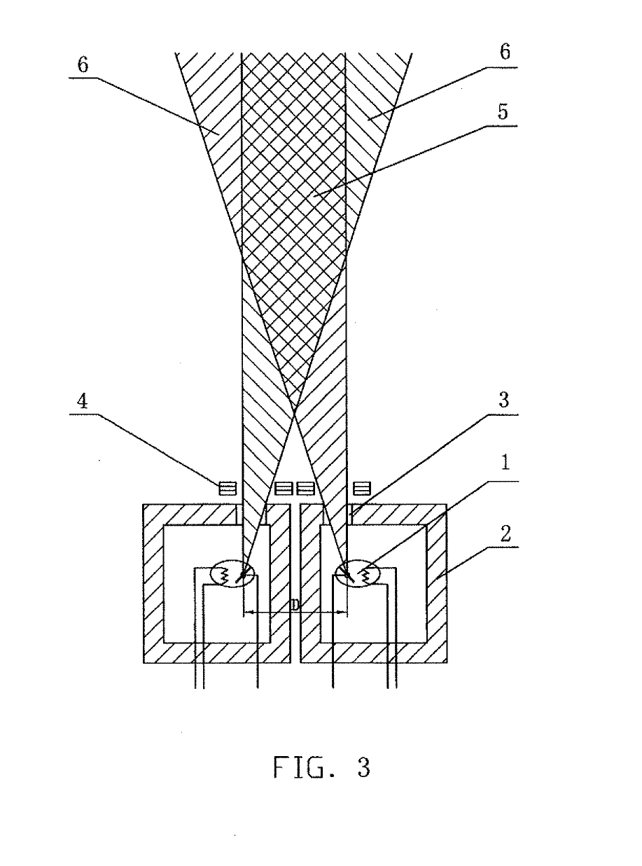

FIG. 4 is a perspective view illustrating structure of three-tube X-ray generator for achieving stereoscopic imaging effect. The difference among FIGS. 1-3 is that this kind of generator has one more X-ray tube and three tubes totally. Therefore, three different angle stereo vision images can be gain without rotating the X-ray generator;

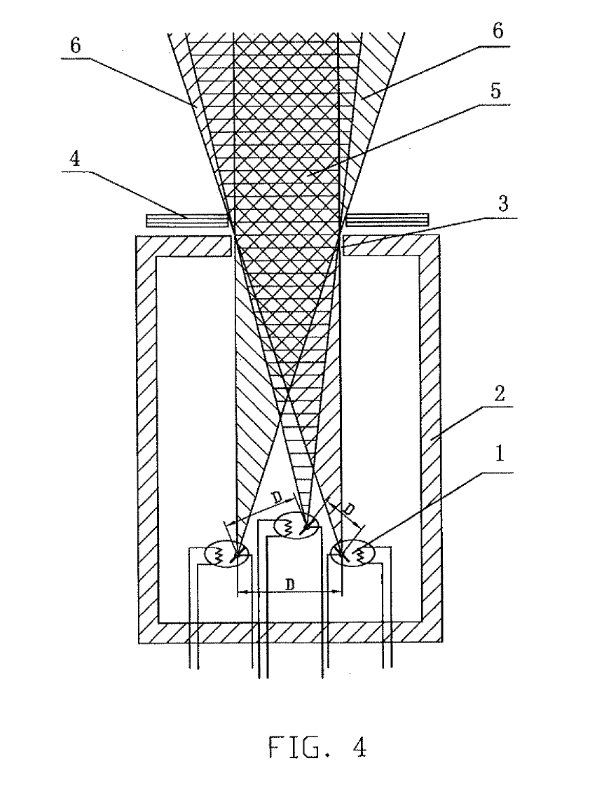

FIG. 5 is a perspective view illustrating structure of single-tube and double-focus type X-ray generator for achieving stereoscopic imaging effect;

FIG. 6 is a perspective view illustrating structure of single-tube, double-focus and double-anode type X-ray generator for achieving stereoscopic imaging effect. The difference between FIG. 6 and FIG. 5 is that FIG. 6 adopts two rotating anodes. High energy electron beam from cathode bombards two rotating anodes alternately to form two anode focuses and future form two X-ray source. The structure in FIG. 5 has single rotating anode, high energy electron beam from cathode bombards two fixing positions in the same rotating anodes alternately to form two anode focuses in the same rotating anodes and future form two X-ray sources;

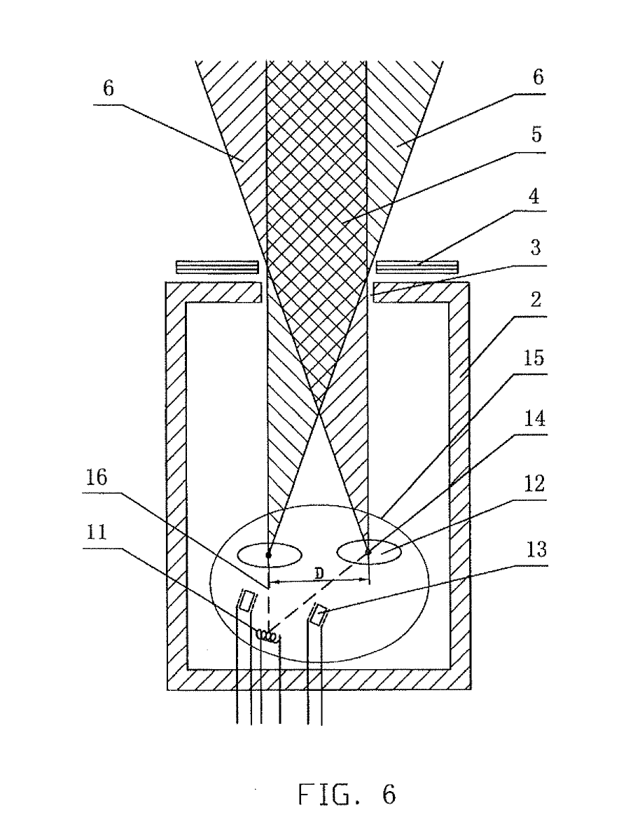

FIG. 7 is a perspective view illustrating structure of single-tube, three-focus type X-ray generator for achieving stereoscopic imaging effect. The difference between FIG. 7 and FIG. 5 is that FIG. 7 adopts three focuses, high energy electron beam from cathode bombards three fixing positions in the same rotating anodes alternately to form three anode focuses in the same rotating anodes and future form three X-ray sources. Data from any two focuses can form stereo vision image, so three different angle stereo vision images can be gain without rotating the X-ray generator;

FIG. 8 is a perspective view illustrating structure of single-tube, two-focus and double-cathode type X-ray generator for achieving stereoscopic imaging effect. The difference between FIG. 8 and FIG. 5 is that FIG. 8 has two cathodes; the high energy electron beam from two cathodes bombards the corresponding positions in the rotating anode to form two anode focuses in the same rotating anode and future form two X-ray sources;

FIG. 9 is a perspective view illustrating structure of single-tube, three-focus and three-cathode type X-ray generator for achieving stereoscopic imaging effect. The difference between FIG. 9 and FIG. 8 is that FIG. 9 adopts three cathodes, high energy electron beam from three cathodes bombards three fixing positions in the same rotating anodes alternately to form three anode focuses in the same rotating anodes and future form three X-ray sources. Data from any two focuses can form stereo vision image, so three different angle stereo vision images can be gain without rotating the X-ray generator;

FIG. 10 is a perspective view illustrating for working principle of real time dynamic stereo vision image diagnose interventional therapy apparatus which install single-tube and two-focus type X-ray generator for achieving stereoscopic imaging effect.

FIG. 11 is a perspective view illustrating for working principle of real time dynamic stereo vision image diagnose interventional therapy apparatus which install single-tube and two-focus type X-ray generator for achieving stereoscopic imaging effect. The difference between FIG. 11 and FIG. 10 is that in FIG. 11, doctors can observe stereo vision image directly on the display of stereo vision display system without stereo vision glasses;

FIG. 12 is a perspective view illustrating for working principle of real time dynamic stereo vision image digital X-ray perspective apparatus which install single-tube and three-focus type X-ray generator for achieving stereoscopic imaging effect;

FIG. 13 is a perspective view illustrating for working principle of X-ray real time dynamic stereo vision digital intestines and stomach machine which install double-tube type X-ray generator for achieving stereoscopic imaging effect;

FIG. 14 is a perspective view illustrating for working principle of X-ray real time dynamic stereo vision image direct radiography machine which install three-tube type X-ray generator for achieving stereoscopic imaging effect; and

FIG. 15 is a perspective view illustrating two-eye type stereo vision.

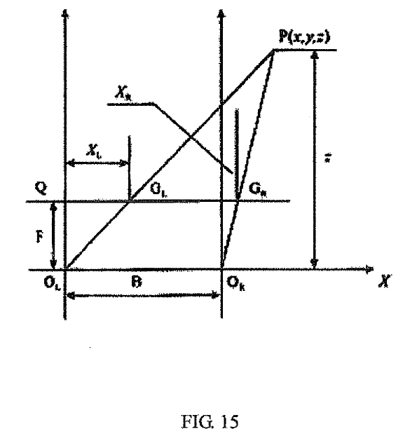

In these figures, 1 denotes X-ray tube, 2 denotes shielding case, 3 denotes window, 4 denotes grating, 5 denotes stereovision data acquisition area, 6 denotes non-stereovision data acquisition area, 11 denotes cathode, 12 denotes anode, 13 denotes deflection electrode, 14 denotes anode focus, 15 denotes vacuum case, 16 denotes high energy electron beam, 101 denotes X-ray generator for achieving stereoscopic imaging effect, 111 denotes data acquisition system, 112 denotes computer data processing system, 113 denotes stereo image display system, 114 denotes manipulator, 115 denotes electrical source and assistant equipment, 116 denotes patient, 117 denotes operation table, 118 denotes doctor, 119 denotes stereo vision glasses.

### DETAILED DESCRIPTION OF THE INVENTION

### Example 1

Double-Tube Type X-Ray Device for Achieving Stereoscopic Imaging Effect

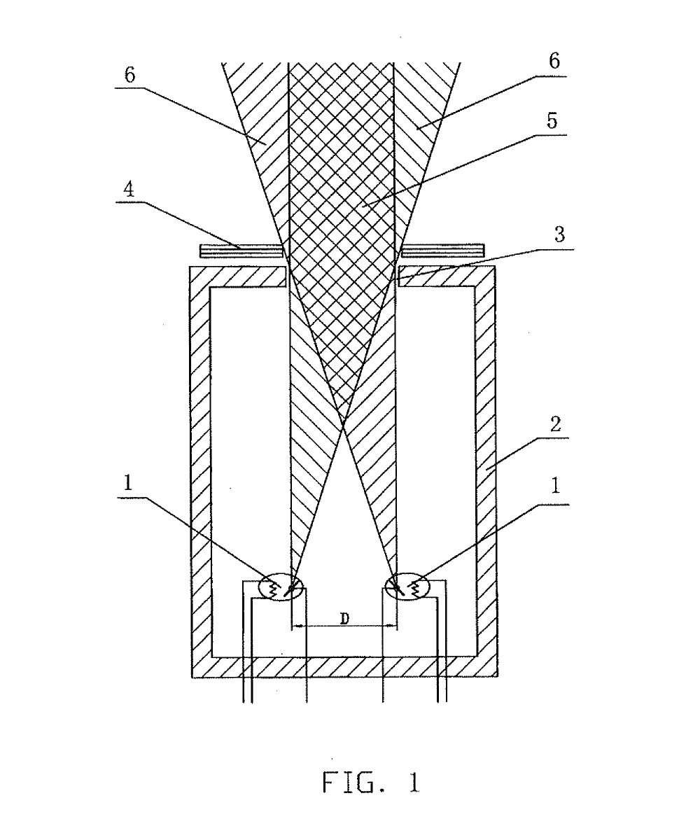

Refer to FIG. 1, in this example, two X-ray tubes 1 may adopt rotating anode tube, the space between the anode focus of the two tubes keeps 9, space D is from 40 mm to 90 mm, the optimum value is 58 mm to 72 mm. the two X-ray tubes 1 are mounted in shielding case 2. X-ray window 3 is mounted on shielding case 2, grating 4 controls the size of X-ray beam.

In the help of electrical source and assistant equipment, the electron beam from cathode 11 which is controlled by deflection electrode 13 bombards anode focus 14 to emit X-ray on two anode focuses alternately. The region which is on outside the shielding case 2 and covered by the X-ray beam which is emitted from two focuses 14 is stereovision data acquisition area 5, and the region which is covered by the X-ray beam which is emitted from only one focus 14 is non-stereovision data acquisition area 6.

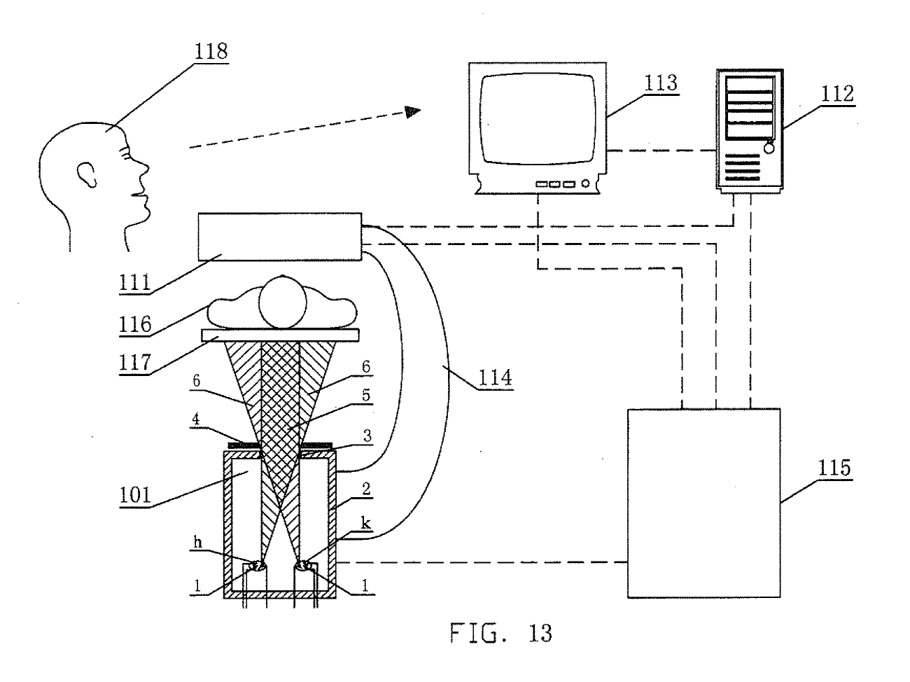

As shown in FIG. 13, as a matter of convenience, X-ray tube 1 in the left of the double-tube type X-ray device for achieving stereoscopic imaging effect 101 is marked as h, the right X-ray tube 1 is marked as k, the space between the anode focus of tube h and the anode focus of tube k is marked as D. The size of the space D may be controlled by electromechanical device; the value is from 40 mm to 90 mm and usually is 65 mm±2 mm.

The X-ray which emitted from the double-tube type X-ray device for achieving stereoscopic imaging effect 101 is controlled by grating 4 to adjust the section which is covered by X-ray beam and control imaging view. X-ray passes through the operation table 117 and patient 116, and then shoots the data acquisition system 111, the data acquisition system 111 is mounted on the section which is covered by X-ray beam and used to acquire image data.

X-ray data acquisition system 111 marks the data which is formed by X-ray beam emitted from tube h as HI data team and the data which is formed by X-ray beam emitted from tube k as K data team, thereinto, the data which is formed by X-ray beam emitted from tube h and noted in stereovision data acquisition area 5 is marked as H5 and the data which is formed by X-ray beam emitted from tube k and noted in stereovision data acquisition area 5 is marked as KS. Data H5 and K5 are associated data which conforms to the requirement for achieving stereoscopic imaging effect of the human beings, data HIS and K5 which are treated by computer data processing system form stereo image in stereo image display system 113 by relief television technique and stereo film technique.

There are many manners for forming stereo image in stereo image display system 113. There are mainly two kinds of mature technologies. One kind is that two images of a pair of parallax signals appear on the screen at the same time, and then the stereo vision is gained by seeing the two images, such as dual channel polarizing image separating relief television technique and complementary color stereo image separating television technique. Another kind is that two images of a pair of parallax signals appear on the screen alternately, and then the stereo vision is gained by seeing the images at different time, such as time-division relief television technique. Furthermore, the new stereo display unit may also be applied. It can import left and right images at different or same time and adopt optical technology to make the left and right image project onto eyes, so the stereo image can be seen on the screen without glasses.

Electrical source and assistant equipment 115 can provide power for the whole machine and control automated. For example, the movement of the manipulator 114 can gain real time stereo vision imaging effect of different angles and different position, which make operation convenience for doctor 118.

### Example 2

Single-Tube and Double-Focus Type X-Ray Device for Achieving Stereoscopic Imaging Effect

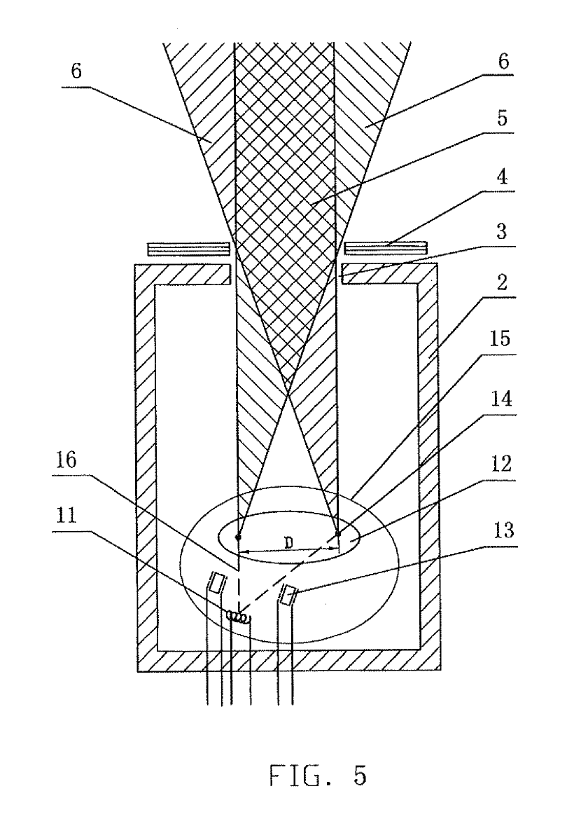

As shown in FIG. 5>this example is great different from example 1.

In this example, the single-tube and double-focus type X-ray device for achieving stereoscopic imaging effect is adopted. Its basic structure principle has the similar part to the routine rotating anode tube in existing technique, comprising cathode 11 and anode 12, but it has a defection electrode 13 which can control the directional of the cathode electron beam. The electron beam from cathode 11 which be controlled by deflection electrode 13 may bombards focus 14 on anode 12 to emit X-ray alternately,

The space between two focuses 14 keeps D, the value of D is from 40 mm to 90 mm, the optimal value is from 58 mm to 72 mm. The position of anode focus 14 on anode 12 can be adjusted by adjusting deflection electrode 13, and the space D between two focuses 14 is controlled at 65 mm±2 mm.

Cathode 11, anode 12 and deflection electrode 13 are set inside the vacuum case 15, and then the vacuum case is set inside the shielding case 2. X-ray window 3 is mounted on shielding case 2, and the size of X-ray beam is controlled by grating 4.

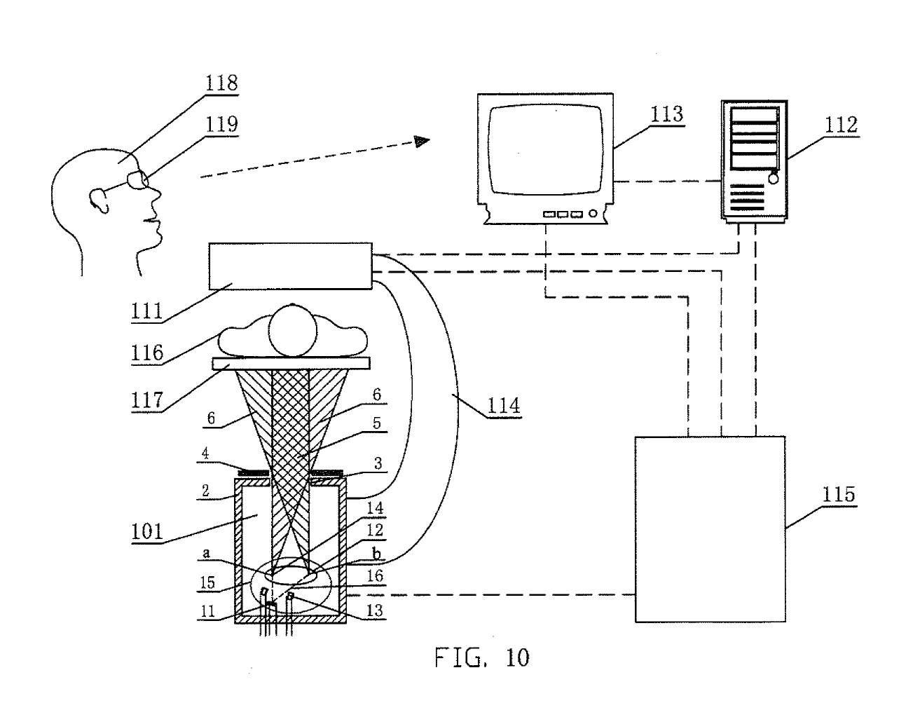

As shown in FIG. 10, In the help of electrical source and assistant equipment, the electron beam from cathode 11 which is controlled by deflection electrode 13 bombards anode focus 14 to emit X-ray on two anode focuses alternately. The region which is on outside the shielding case 2 and covered by the X-ray beam which is emitted from two focuses 14 is stereovision data acquisition area 5, and the region which is covered by the X-ray beam which is emitted from only one focus 14 is non-stereovision data acquisition area 6.

X-ray data acquisition system 111 is installed on the region which is covered by X-ray beam, the data which is formed by X-ray which is emitted from the same focus in stereovision data acquisition area 5 is noted as one team. For the clear expression, the left anode focus is marked as a, and the right anode focus is marked as b, the data which is formed by X-ray beam emitted from focus a and noted in stereovision data acquisition area 5 is marked as A5 and the data which is formed by X-ray beam emitted from focus b and noted in stereovision data acquisition area 5 as B5. Data A5 and B5 are associated data which conforms to the requirement for achieving stereoscopic imaging effect of the human beings, data A5 and B5 which are treated by computer data processing system 112 form stereo image in stereo image display system 113 by relief television technique and stereo film technique.

### Example 3

Single-Tube and Three-Focus Type X-Ray Device for Achieving Stereoscopic Imaging Effect

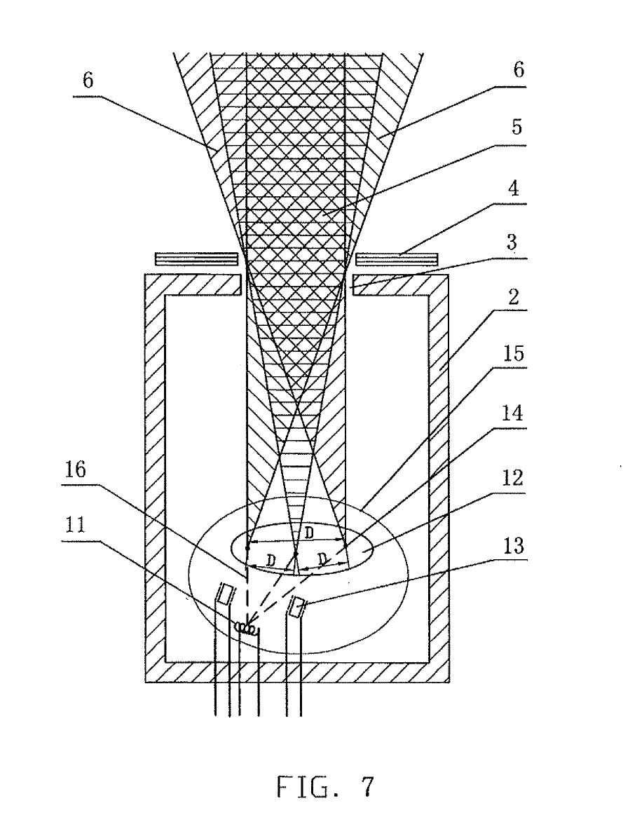

As is shown in FIG. 7, the basic principle in this example is similar to example 2, comparing FIG. 7 and FIG. 5. Different from example 2, it adopts three focuses, the high energy electron beam 16 from cathode 11 bombards three different position on the same rotating anode 12 alternately, forming three anode focuses 14 on single rotating anode, and the X-ray is emitted from the three anode focuses 14 alternately.

If the space of any two of the three focuses all conforms to the requirement for achieving stereoscopic imaging effect of the human beings, three pairs of data which can be gained conforms to the requirement for achieving stereoscopic imaging effect of the human beings, and corresponding three pairs of stereo vision image can be formed. Three different angles of stereo vision image can be gained by this kind of technique without rotating the X-ray generator If only the doctors switch the images, they can observe the real time stereo dynamic images from three different angles, which is very convenience for diagnosing and operation.

### Example 4

Single-Tube, Double-Cathode and Double-Focus Type X-Ray Device for Achieving Stereoscopic Imaging Effect

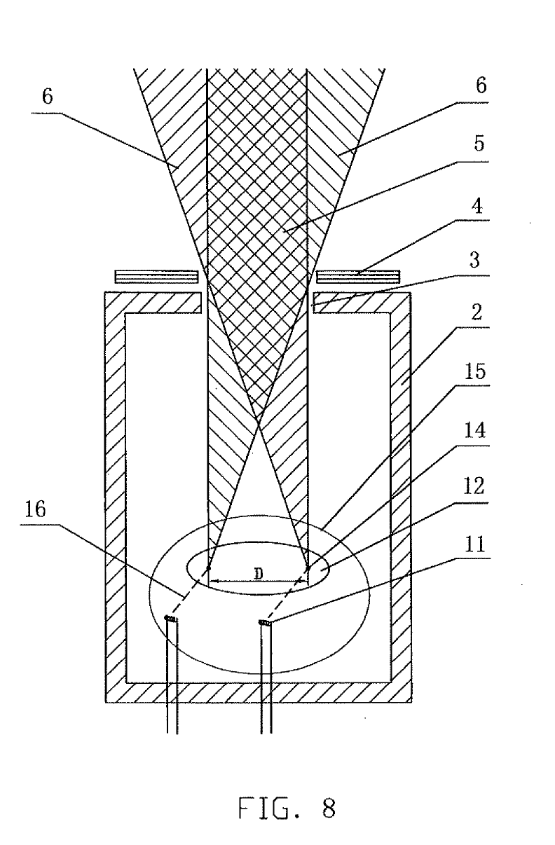

As is shown in FIG. 8, the basic principle in this example is similar to example 2, comparing FIG. 8 to FIG. 5.

Different from Example 2, Example 4 adopts two cathode 11, of which high energy electron beam 16 bombards corresponding position on the same rotating anode 12. Two anode focuses 14 are formed on the same rotating anode 12 and two X-ray sources are formed. A pair of data which conforms to the requirement for achieving stereoscopic imaging effect of the human beings is provided to form stereo vision image.

### Example 5

Single-Tube, Three-Cathode and Three-Focus Type X-Ray Device for Achieving Stereoscopic Imaging Effect

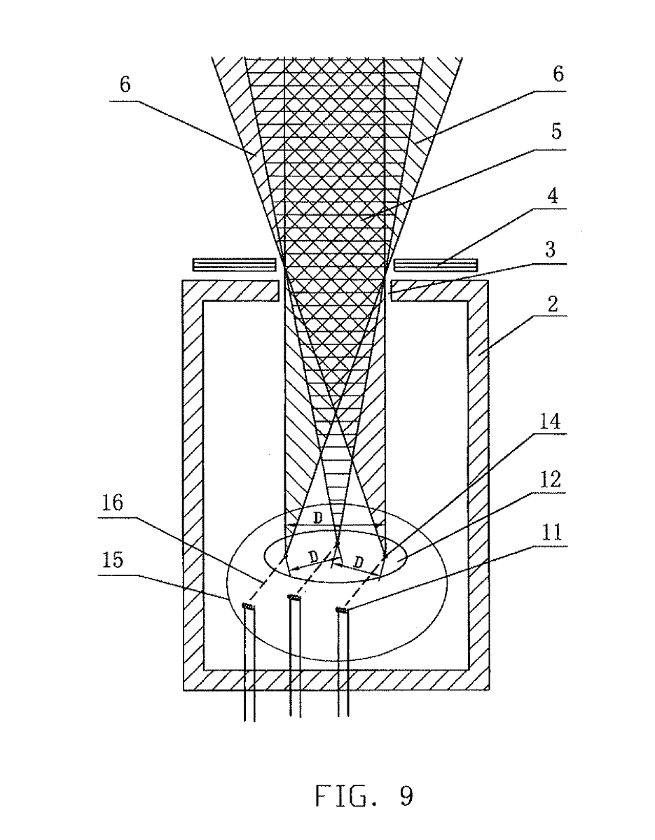

As is shown in FIG. 9, the basic principle in this example is similar to example 4, comparing FIG. 8 to FIG. 9

Different from example 4, Example 5 adopts three cathodes 11, the high energy electron beam 16 from three cathodes 11 bombards corresponding position on the same rotating anode 12, forming three anode focuses 14 on single rotating anode, and three X-ray sources. If the space of any two of the three focuses all conforms to the requirement for achieving stereoscopic imaging effect of the human beings, three pairs of data which can be gained conforms to the requirement for achieving stereoscopic imaging effect of the human beings, and corresponding three pairs of stereo vision image can be formed. Three different angles of stereo vision image can be gained by this kind of technique without rotating the X-ray generator If only the doctors switch the images, they can observe the real time stereo dynamic images from three different angles, which is very convenience for diagnosing and operation.

### Example 6

Real Time Dynamic Stereo Vision Image Diagnose Interventional Therapy Apparatus

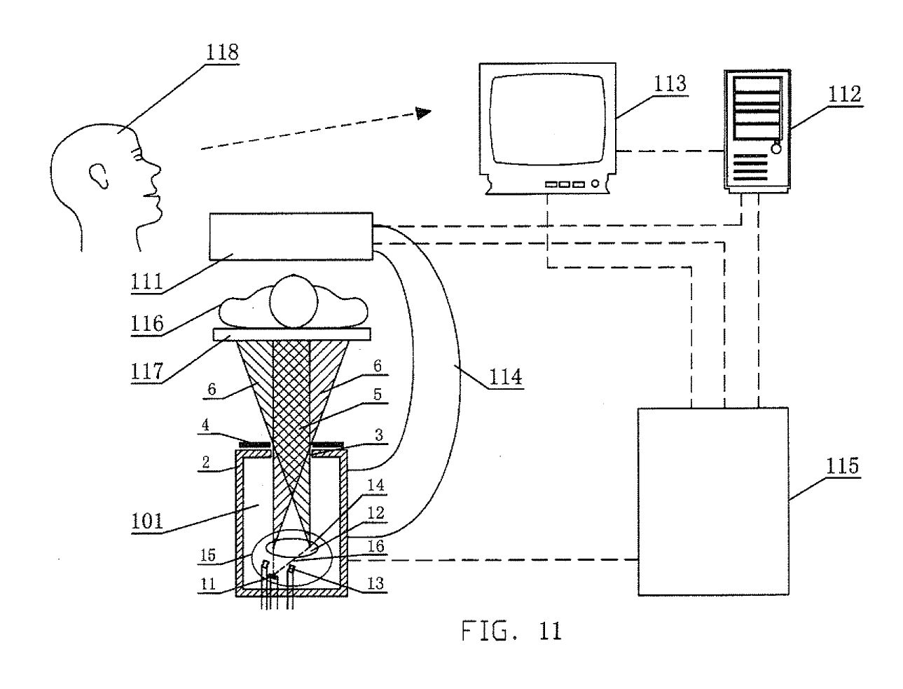

As is shown in FIG. 10 and FIG. 11, in this example, the single-tube and double-focus type X-ray device for achieving stereoscopic imaging effect is used as the X-ray source of real time dynamic stereo vision image diagnose interventional therapy apparatus

In the help of electrical source and assistant equipment, the electron beam from cathode 11 which is controlled by deflection electrode 13 bombards anode focus 14 to emit X-ray on two anode focuses alternately. A pair of date which conforms to the requirement for achieving stereoscopic imaging effect of the human beings is gained by X-ray data acquisition system 111 in stereovision data acquisition area 5. The pair of data is treated by computer data processing system 112 to form stereo image in stereo image display system 113 by relief television technique and stereo film technique.

This kind of medical X-ray equipment can generate stereo vision. In the perspective mode, interventional physician can see tridimensional skull as crystal, blood vessel and bones. The risk of operation is low.

In this example, the relief television technique and stereo film technique, such as time-division relief television technique, dual channel polarizing image separating relief television technique and complementary color stereo image separating television technique. The doctors 118 need stereo glasses 113 to observe stereo vision image in stereo image display system. See FIG. 10.

The new stereo display unit may also be applied. It can import left and right images at different or same time and adopt optical technology to make the left and right image project onto eyes, so the stereo image can be seen on the screen without glasses. See FIG. 11.

### Example 7

Real Time Dynamic Stereo Vision Image Digital X-Ray Perspective Apparatus

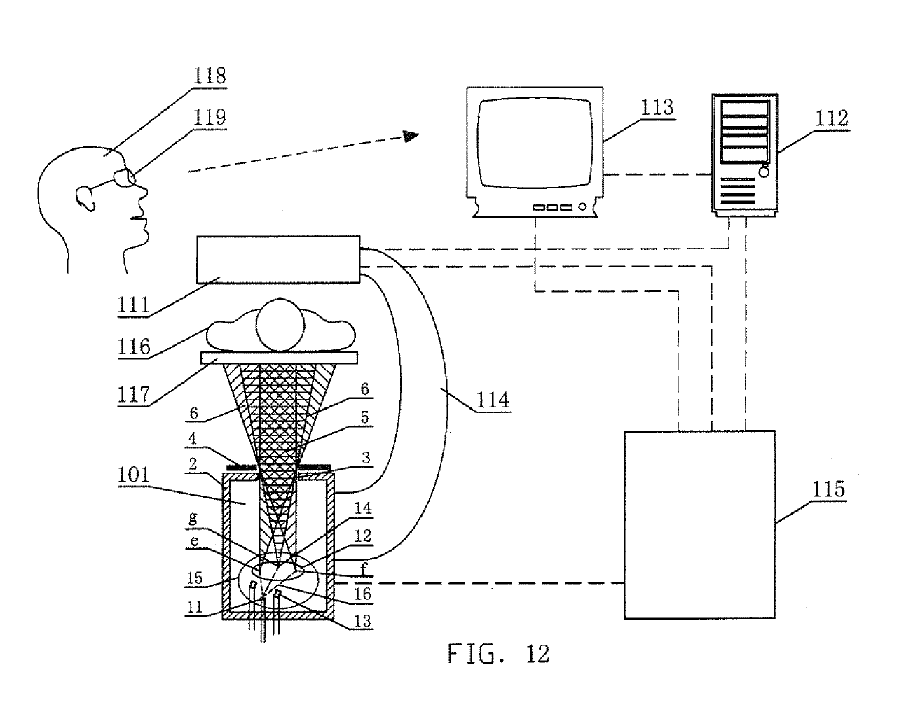

As is shown in FIG. 12, in this example, the single-tube and three-focus type X-ray device for achieving stereoscopic imaging effect is used as the X-ray source of real time dynamic stereo vision image digital X-ray perspective apparatus.

In the help of electrical source and assistant equipment, the high energy electron beam 16 from cathode 11 bombards three corresponding positions on the same rotating anode 12 alternately, forming three anode focuses 14 on single rotating anode and three X-ray sources.

If the space of any two of the three focuses all conforms to the requirement for achieving stereoscopic imaging effect of the human beings, three pairs of data which can be gained conforms to the requirement for achieving stereoscopic imaging effect of the human beings, and corresponding three pairs of stereo vision image can be formed. Three different angles of stereo vision image can he gained by this kind of technique without rotating the X-ray generator. If only the doctors switch the images, they can observe the real time stereo dynamic images from three different angles. In the perspective mode, the surgical doctor can see human tissues which have third dimension. With this equipment, the diagnosis is more exactitude. See FIG. 12.

### Example 8

X-Ray Real Time Dynamic Stereo Vision Digital Intestines and Stomach Machine.

As is shown in FIG. 13, in this example, the double-tube type X-ray device for achieving stereoscopic imaging effect is used as the X-ray source of X-ray real time dynamic stereo vision digital intestines and stomach machine.

In the help of electrical source and assistant equipment, the electron beam from cathode 11 which is controlled by deflection electrode 13 bombards anode focus 14 to emit X-ray on two anode focuses alternately. The region which is on outside the shielding case 2 and covered by the X-ray beam which is emitted from two focuses 14 is stereovision data acquisition area 5, and the region which is covered by the X-ray beam which is emitted from only one focus 14 is non-stereovision data acquisition area 6

X-ray tube 1 in the left of the double-tube type X-ray device for achieving stereoscopic imaging effect 101 is marked as h, the right X-ray tube 1 is marked as k, and the space between the anode focus of tube h and the anode focus of tube k is marked as D. The size of the space D may be controlled by electromechanical device; the value is from 40 mm to 90 mm and usually is 65 mm±2 mm.

The size of the X-ray emitted from double-tube type X-ray device for achieving stereoscopic imaging effect 101 is controlled by the grating 4, thereby the region covered by the X-ray is regulated, and form the visual field. The X-ray goes through the operation table 117 and the patient 116, and goes to the data acquisition system 111, and the data acquisition system 111 receives the image data from the region covered by the X-ray.

The X-ray data acquisition system 111 marks the data which is formed by X-ray beam emitted from tube h as H data team and the data which is formed by X-ray beam emitted from tube k as K data team, thereinto, the data which is formed by X-ray beam emitted from tube h and noted in stereovision data acquisition area 5 is marked as H5 and the data which is formed by X-ray beam emitted from tube k and noted in stereovision data acquisition area 5 is marked as KS. Data H5 and K5 are associated data which conforms to the requirement for achieving stereoscopic imaging effect of the human beings, data 145 and K5 which are treated by computer data processing system form stereo image in stereo image display system 113 by relief television technique and stereo film technique.

The electrical source and assistant equipment 115 can provide power for the whole machine and control automated. For example, the movement of the manipulator 114 can gain real time stereo vision imaging effect of different angles and different position, which make operation convenience for doctor 118.

### Example 9

X-Ray Real Time Dynamic Stereo Vision Image Direct Radiography (DR) Machine.

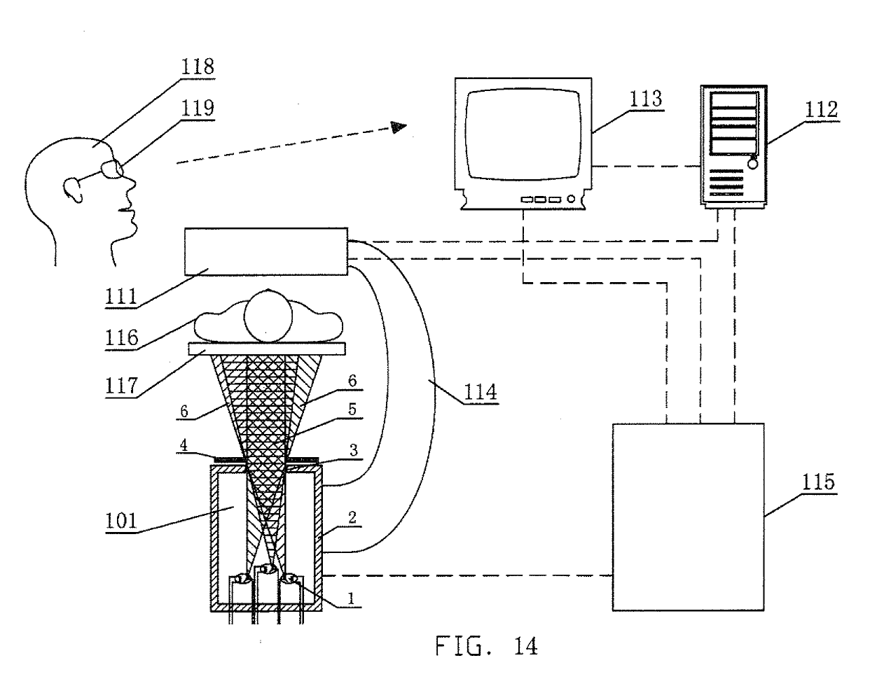

As is shown in FIG. 14, in this example, the three-tube type X-ray device for achieving stereoscopic imaging effect is used as the X-ray source of X-ray real time dynamic stereo vision image direct radiography machine.

In the help of electrical source and assistant equipment, the three X-ray tubes emit X-ray alternately; a pair of date which conforms to the requirement for achieving stereoscopic imaging effect of the human beings is gained by X-ray data acquisition system 111. The pair of data is treated by computer data processing system 112 to form stereo image in stereo image display system 113 by relief television technique and stereo film technique. Three different angles of stereo vision image can be gained by this kind of technique without rotating the X-ray generator. If only the doctors switch the images, they can observe the real time stereo dynamic images from three different angles. In the perspective mode, the surgical doctor can see human tissues which have third dimension. With this equipment, the diagnosis is more exactitude.

1. An X-ray generator (101) for achieving stereoscopic imaging effect and an X-ray device with stereoscopic imaging effect, characterized in that the X-ray generator deployed to emit X-ray alternately from two positions the space of which conforms to the requirement for achieving stereoscopic imaging effect of the human beings, and the X-ray device use the X-ray generator (101) as the X-ray source.

2. The X-ray generator for achieving stereoscopic imaging effect according to claim 1, wherein the space of the two positions is close to papillary distance and the space is 40 mm to 90 mm, and the optimum space is 58 mm to 72 mm.

3. The X-ray generator for achieving stereoscopic imaging effect according to claim 1, wherein said X-ray generator comprises at least two X-ray tubes (1) which emit X-ray alternately, and the space (D) of the X-ray tubes (1) is 40 mm to 90 mm, and the optimum space is 58 mm to 72 mm.

4. The X-ray generator for achieving stereoscopic imaging effect according to claim 1, wherein said X-ray generator comprises cathode (11), anode (12), deflection electrode (13), and at least two anode focuses (14); the high energy electron beam emitted from cathode (11) and controlled by deflection electrode (13) hits the different anode focuses (14) to emit X-ray alternately; the space (D) of different anode focuses (14) is 40 mm to 90 mm, and the optimum space (D) is 58 mm to 72 mm.

5. The X-ray generator for achieving stereoscopic imaging effect according to claim 4, wherein said X-ray generator has three anode focuses (14), deployed to emit X-ray alternately from two or three anode focuses (14).

6. The X-ray generator for achieving stereoscopic imaging effect according to claim 4, wherein said X-ray generator has at least two cathodes ( 11), the high energy electron beam from each cathode (11) hits the corresponding anode focuses (14) alternately, and the X-ray is emitted from anode focus (14) alternately.

7. The stereoscopic imaging effect medical X-ray device according to claim 1, comprising a digital subtraction angiography for achieving stereoscopic imaging effect, which applies X-ray generator for achieving stereoscopic imaging effect as the X-ray source for digital subtraction angiography.

8. The stereoscopic imaging effect medical X-ray device according to claim 1, comprising a digital gastrointestinal apparatus for achieving stereoscopic imaging effect, which applies X-ray generator for achieving stereoscopic imaging effect as the X-ray source for digital gastrointestinal apparatus.

9. The stereoscopic imaging effect medical X-ray device according to claim 1, comprising real time dynamic stereo vision image digital X-ray perspective apparatus, wherein the real time dynamic stereo vision image digital X-ray perspective apparatus use the X-ray generator for achieving stereoscopic imaging effect as X-ray source.

10. The stereoscopic imaging effect medical X-ray device according to claim 1, comprising a digital X-ray perspective apparatus for achieving stereoscopic imaging effect, which applies X-ray generator for achieving stereoscopic imaging effect as the X-ray source for digital X-ray perspective apparatus.
